(19) United States
(12) Patent Application Publication
(10) Pub. No.: US 2023/0094452 A1
(43) Pub. Date: Mar. 30, 2023

(54) MEASURING PHYSIOLOGICAL CHARACTERISTICS

(71) Applicant: HUMA THERAPEUTICS LIMITED, London (GB)

(72) Inventors: Tamir Strauss, London (GB); Emily Binning, London (GB); Danoosh Vahdat, London (GB); Mert Aral, London (GB); Sam Nikbakhtian, London (GB)

(21) Appl. No.: 17/908,852

(22) PCT Filed: Mar. 5, 2020
(86) PCT No.: PCT/GB2021/050554

§ 371 Date: Sep. 1, 2022

(30) Foreign Application Priority Data
Mar. 5, 2020 (GB) 2003223.1

(51) Int. Cl. A61B 5/11; A61B 5/00

(52) U.S. Cl. CPC A61B 5/112; A61B 5/1117; A61B 5/6801; A61B 5/1123; A61B 5/7275

(57) ABSTRACT

A data processing system comprising a processing device configured to collect sets of sensed data representing motion characteristics of a user's gait at a first time and at a second time subsequent to the first time, to process each set of sensed data to form a respective information set dependent on the user's gait at the first and second times, and to compare the first information set with one of (i) the second information set and (ii) a predetermined reference data set in accordance with a predetermined algorithm to identify a condition or change in condition of the user.

This invention relates to measuring one or more physiological characteristics of a subject and making predictions based on those measurements: for example predictions of the subject's chance of being predisposed to certain medical conditions.

It is known for portable devices such as mobile/cellular phones or wearable devices such as smart watches or dedicated sensors to measure aspects of a user's behaviour or physiological function. For example, a portable device may estimate the number of steps that a user carrying the device has made in a given period of time, or a wearable device may sense a user's heart rate, blood pressure or rate of movement. It is known to make estimates of the user's health using these measurements. For example, it is possible to estimate whether it might be desirable for the user to be more or less active, or to estimate the user's locomotive efficiency. A limitation of these mechanisms is that it is difficult to establish, when the measurements are being taken, whether there are any extraneous factors affecting the user which might have a bearing on the accuracy of the measurement. For illustration, consider a system that is attempting to estimate a user's locomotive efficiency. The user's actual efficiency might depend on whether they are carrying a substantial load in addition to their bodyweight, but the system might be unable to sense whether a weight is present. It can thus be difficult to generate reliable estimates of physiological parameters.

Once an estimate of one or more of a user's physiological characteristics is available, it or they may be used to generate predictions of other factors: for example the user's propensity to a medical condition or the status of an existing one. It would be desirable to increase the range of predictions that can be made in this way. It would be desirable to increase the accuracy of such predictions.

It is known to gather data from dedicated activity sensors such as sports watches and stride-sensing accelerometers and to analyse the data from those sensors to estimate concrete characteristics of the gait of a person carrying the sensors. For example, it is known to estimate the user's stride length, stride frequency or the amount of upward motion in the user's stride. This data is then used to estimate factors such as the user's efficiency at running.

According to one aspect there is provided a data processing system comprising a processing device configured to collect sets of sensed data representing motion characteristics of a user's gait at a first time and at a second time subsequent to the first time, to process each set of sensed data to form a respective information set dependent on the user's gait at the first and second times, and to compare the first information set with one of (i) the second information set and (ii) a predetermined reference data set in accordance with a predetermined algorithm to identify a condition or change in condition of the user.

The condition may be one of the following: a level of pain and a level of recovery from surgery, a level of fall risk and a likelihood of the user suffering from a predetermined disease.

The sensed data may comprise one of translational acceleration, rotational acceleration, translational speed and rate of rotation.

According to a second aspect there is provided a data processing system comprising a processing device configured to collect sensed data representing the acceleration characteristics of a user's gait and associated locations and to process that data in accordance with a predetermined algorithm to (i) estimate instances in the acceleration characteristics that are indicative of uneven motion and (ii) identify locations within a predetermined distance of which more than a predetermined number of such instances have occurred and to output an indication of such locations.

The system may be configured to identify the said instances as being instances for which the acceleration data has either (i) a relatively high correlation with reference data representing uneven motion or (ii) a relatively low correlation with reference data representing even motion.

According to a third aspect there is provided a data processing system comprising a processing device configured to:
at a first time, collect sensed data representing motion characteristics of a first user gait and thereby form reference data representing a reference state of the user's gait; and
at a second time subsequent to the first time, collect sensed data representing motion characteristics of a second user gait, compare that data with the reference data and in dependence on that comparison form an indication of a level of confidence that the first user gait and the second user gait are of the same individual.

The system may be configured to receive further sensed data, and to discard that data if the said level of confidence is below a predetermined threshold.

The system may be configured to govern access to a resource and to determine whether to provide access to that resource in dependence on whether the said level of confidence is above a predetermined threshold.

The system may be a mobile phone having a major display face and the system may be configured to preferentially sense the sensed data when a normal to the major face is substantially horizontal.

The system may be configured to preferentially sense the sensed data when a normal to the major face is within a predetermined angle of horizontal.

The data processing system may comprise one or more sensors for sensing the said sensed data.

The sensor(s) may be packaged so as to be hand portable or wearable.

At least one sensor may be provided with one of: a lanyard, a wrist strap, within an item of clothing, a body implant and an adhesive layer whereby the sensor can be attached to the skin of a user.

The system may be configured to aggregate sensed data over a window of 3 seconds or less to form an aggregated measurement and to apply the aggregated measurement as an input to the said algorithm.

The system may be configured to apply a low-pass filter to sensed data to form a filtered measurement and to apply the filtered measurement as an input to the said algorithm.

The system may be configured to process at least some of the sensed data to estimate its correlation to predetermined data indicative of an activity of a user and to, in implementing the said algorithm, apply a greater weight to data sensed at a time when that correlation is high.

The present invention will be described by way of example with reference to the accompanying drawings. In the drawings:

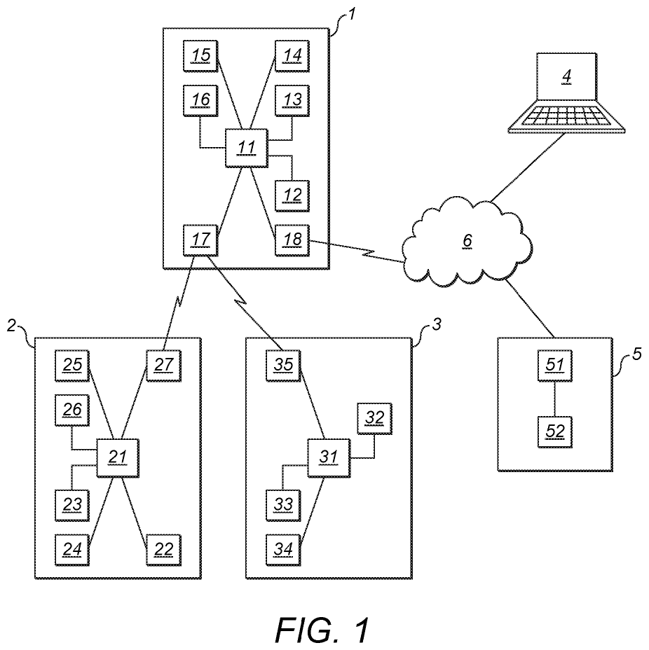

FIG. 1 is a schematic diagram of a system for measuring physiological characteristics of a user and making predictions in dependence on those measurements.

FIG. 1 shows a system comprising a portable device 1, a wearable device 2 having an enhanced user interface, a wearable device 3 having a limited user interface, a user terminal 4 and a server 5. The portable device 1 can be carried by a subject or user. The wearable devices 2, 3 can be worn by the subject. The portable device 1 and the wearable devices 2, 3 comprise sensors for sensing information about the behaviour or functioning of the subject. That information can be processed locally by the portable device 1 and/or the wearable devices 2, 3, or remotely by the terminal 4 or the server 5, to generate estimates of the chance of certain future occurrences. Information about the estimated chances can then be presented to the user, for instance by the portable device 1, wearable device 2 or terminal 4. Terminal 4 may be used by another individual: for example a healthcare professional.

In more detail, portable device 1 is a portable device capable of data collection and/or processing. It can also present information to a user. Device 1 comprises a processor 11, a memory 12, a display 13, one or more user input units 14 and one or more sensors 15, 16. The memory stores in a non-transient way code executable by the processor to cause it to perform the functions described of it herein. The sensors are coupled to the processor to provide data they sense to the processor. The display is coupled to the processor so that the processor can control the display to output desired information. The user inputs are coupled to the processor so that the processor can receive information that is input by a user and can then perform processing in dependence on that data. The user inputs could, for example be press-switches, or the inputs could be integrated with the display 13 as in the case of a touch screen. The sensors could be of any suitable type. Examples will be given below, without limitation. The device 1 also comprises communication interfaces such as a short-range interface 17 and a long-range interface 18. Interface 17 may support protocols such as IEEE 802.11, Bluetooth and ANT. Interface 18 may support protocols such as 4G, 5G and Ethernet. One or both of the interfaces may support a wireless protocol. Device 1 could, for example, be a dedicated data collection device, a mobile or cellular phone or a tablet computer.

Wearable device 2 is a wearable device capable of data collection and/or processing. It can also present information to a user. Device 2 comprises a processor 21, a memory 22, a display 23, one or more user input units 24 and one or more sensors 25, 26. The memory stores in a non-transient way code executable by the processor to cause it to perform the functions described of it herein. The sensors are coupled to the processor to provide data they sense to the processor. The display is coupled to the processor so that the processor can control the display to output desired information. The user inputs are coupled to the processor so that the processor can receive information that is input by a user and can then perform processing in dependence on that data. The user inputs could, for example be press-switches, or the inputs could be integrated with the display 23 as in the case of a touch screen. The sensors could be of any suitable type. Examples will be given below, without limitation. The device 2 also comprises a communication interface 27. It may support long or short-range communication. It may support wired or wireless communication. Conveniently it supports a communication protocol in common with interface 17 and/or 18 of device 1, allowing devices 1 and 2 to intercommunicate. Interface 27 may support protocols such as IEEE 802.11, Bluetooth, ANT, 4G, 5G and Ethernet. Device 2 may be wearable by being adapted for attachment to a user's body. Examples of ways in which it may be attached include by features such as a wrist strap, a lanyard, within an item of clothing, implant or an adhesive patch. Device 2 may comprise such features or they may be provided as accessories to device 2.

Wearable device 3 is a wearable device capable of data collection and processing. It cannot directly present information to a user. Device 3 comprises a processor 31, a memory 32 and one or more sensors 33, 34. The memory stores in a non-transient way code executable by the processor to cause it to perform the functions described of it herein. The sensors are coupled to the processor to provide data they sense to the processor. The display is coupled to the processor so that the processor can control the display to output desired information. The user inputs are coupled to the processor so that the processor can receive information that is input by a user and can then perform processing in dependence on that data. The sensors could be of any suitable type. Examples will be given below, without limitation. The device 3 also comprises a communication interface 35. It may support long or short-range communication. It may support wired or wireless communication. Conveniently it supports a communication protocol in common with interface 17 and/or 18 of device 1, allowing devices 1 and 3 to intercommunicate. Interface 35 may support protocols such as IEEE 802.11, Bluetooth, ANT, 4G, 5G and Ethernet. Device 3 may be wearable by being adapted for attachment to a user's body. Examples of ways in which it may be attached include by features such as a wrist strap, a lanyard, within an item of clothing, implant or an adhesive patch. Device 3 may comprise such features or they may be provided as accessories to device 3.

Data sensed by devices 1, 2 and 3 can be transmitted over a network 6 to terminal 4 and/or server 5. FIG. 1 illustrates that device 1 can gather data from devices 2 and 3 and can then forward that data over network 6, but other arrangements are possible. For example, one or both of devices 2, 3 could communicate directly over network 6.

In practice any one, two or three of devices 1-3 may be used. Multiple sensor devices like device 2, or like device 3 may be used, for example to sense respective aspects of function (e.g. physiological function or biomechanical function). In one convenient arrangement only device 1 is used, with that device being a smartphone having multiple sensors for (e.g.) longitudinal and/or rotational acceleration which may be integrated to give estimates of (e.g.) horizontal distance, height and/or cumulative rotation.

Server 5 can process data received from devices 1, 2 and 3, for example to apply predetermined algorithms to that data and optionally to other pre-stored data so as to form estimates of future events in the manner to be described below. To that end, server 5 comprises a processor 51 and a memory 52. Memory 52 may comprise one or more solid state memory units and/or one or more disk drives. Memory 52 stores in a non-transient form program code executable by processor 51 to cause it to execute the functions described of it herein. The program code defines algorithms for analysing data sensed by devices 1 to 3, and the processor implements the algorithms. It should be noted that those algorithms may alternatively be implemented at one or more of devices 1, 2 and 3, or at terminal 4, or distributed in any convenient way between one or more of those devices and server 5.

A user of terminal 4 can view data received from devices 1, 2 and 3 and/or the outputs of the algorithms discussed above. Such data and/or outputs can alternatively be viewed on the displays of devices 1 and 2 or in any other convenient way. An application or app running on the processor of device 1 or 2 may interface with the server 5 to receive information and may then present that information to a user. The same application may assist in analysing the sensed data by means of a suitable algorithm, as will be discussed below.

In summary, physiological data about a user can be collected by the sensors of devices 1, 2 and 3. That data can then by processed according to predetermined algorithms by any of devices 1 to 5. The data and/or the outputs of the algorithms can then be presented to a user in any convenient manner.

In the present system, data is collected from one or more sensors as a user carrying the sensor(s) walks. That data is managed (e.g. by storing it). Then the data is pre-processed (e.g. by filtering it to remove outlying values and/or to reduce the amount of data by culling unnecessary samples, or to normalise it based on different ways the device is worn or held). This results in signals of relatively high quality. These signals represent a signature of the user's gait. There is no need to derive data representing macroscopic artefacts of the user's gait, such as stride length, frequency, rotation of torso during stride. The data signature is used to monitor changes in the user's gait over time by comparing a contemporaneously captured signature with a baseline signature captured for the same user at a previous time. Some specific gait types are known to be associated with certain conditions: for example the hemiplegic gait is known to be shown by patients with e.g. stroke, the spastic diplegic gait is known to be shown by patients with e.g. cerebral palsy and the myopathic gait is known to be shown by patients with e.g. muscular dystrophy. In the present system data to map a gait to a classical specific gait type is not captured. Instead, the gait signature information is analysed. This may conveniently be done by a machine learning technique or other artificial intelligence methods. A set of training data may be formed comprising gait signature data captured during the ambulation of a range of subjects, some of whom have not been diagnosed with any relevant medical condition and others of whom have been diagnosed with one or more medical conditions. The training data can be analysed using a suitable machine learning algorithm to form a trained model associating input gait signatures with outputs representing indications of conditions and optionally their status and/or progression. That model may be implemented on a portable device (e.g. device 1) or on a static server 5. Contemporaneously captured gait signatures can be input to the model and the model may then indicate the suspected presence of a condition and optionally its suspected severity.

Non-limiting examples of the sensors 15, 16, 25, 26, 33, 34 include the following: heart rate sensors, e.g. optical heart rate sensors; blood pressure sensors; accelerometers, e.g. three-axis accelerometers; three-axis gyroscopes; temperature sensors; barometric pressure sensors; satellite location receivers for sensing location; sound sensors such as microphones; magnetometers; applied force sensors such as load cells and/or piezoelectric sensors; and radiation detectors.

The operation of the system of FIG. 1 for certain measuring and estimation purposes will now be described. It will be appreciated that the system may sense multiple types of information and process that information in multiple ways, so that it can make multiple types of predictions about a single user. In the descriptions below it should be understood that a user of the system about whom data is to be sensed carries the portable device 1 (e.g. in a pocket or a handbag) and/or wears one or both of devices 2, 3 in any suitable manner. In that way, the device(s) carried or worn by the user can sense data about the behaviour and functioning of that user. The user may carry or wear all of devices 1 to 3 or any subset of them, as is appropriate to the data that is to be sensed.

When a user who is carrying or wearing devices 1-3 is walking, running or otherwise ambulating, a sensor of one of the devices can sense the accelerations associated with that activity. In one example, the sensor may be an accelerometer which can sense accelerations. Those accelerations may be along or about one or more axes and may be distributed over time. The accelerations may include the forward motion of the ambulation, the vertical motion of the ambulation and the lateral motion of the ambulation. They may include rotational accelerations associated with the ambulation. The pattern of accelerations may vary with time over the course of a pace, may vary as between paces of the user's left and right legs or may vary with the time the user has spent ambulating. The user may be moving on a flat surface, but equally could be moving uphill, downhill, up or down stairs and so on. In other examples the sensor could be a gyroscope or a magnetometer. Such sensors can be used to detect different patterns of movement, e.g. vertical movement. The sensed movements, accelerations and/or a pattern of them may be taken to characterise the user's gait for the time being. That information may be stored at one time (e.g. at device 1 or at server 5), and compared with the gait information sensed at a later time. In that way the system can assess whether the user's gait has changed over time.

It has been found that a representative gait signature can be captured by sensing data as a user ambulates over a relatively short time period: for example less than 10 seconds, less than 5 seconds or less than 4 seconds. It has been found that a convenient period over which to sample is between 2 and 4 seconds. One benefit of a relatively short sampling window is that it allows the system to generate many disparate data points quickly, thus improving the accuracy of the solution and the classification rate. An artificial intelligence/machine learning algorithm may be used to identify the data sampling window that yields the most accurate and rapid classification results.

When data has been sensed, it is desirable to apply a low pass filter to that data, e.g. by means of a Fourier transform. This can improve the signal to noise ratio of the data. Sensors whose data can benefit from low pass filtering include accelerometers, gyroscopes and magnetometers. Other noise filtering methods can also be used to improve the quality and fidelity of the signals produced by the sensors before feeding the dataset to the classification algorithm.

It is desirable that the sensing system (e.g. at device 1) selects when to sample data about a user's gait in dependence on information indicative of whether (i) a sensor is being carried by the user and/or (ii) whether the user is currently ambulating. Examples of such information include:
the time of day: a user is typically active at similar times of each day;
the orientation of the device: when a phone is laid flat it is unlikely to be in a user's pocket and may instead be in a user's hand or on a table: gait sensing by a phone may be limited to times when the phone is in a generally upright orientation: for example when a normal to its major display face is within a predetermined angle from horizontal, such as within 10 degrees or within 20 degrees or within 30 degrees of horizontal;
the location of the device and/or its speed and/or its acceleration and/or its pattern of acceleration: for example sensing may be inhibited when the device is moving at a speed greater than walking pace or is moving in a way that is not broadly characteristic of normal walking, for example ascending stairs or turning.

Instead of not sensing gait during the periods indicated above, the device(s) could sense gait data then but discard the sensed data when one or more of the conditions described above apply.

Gait information sensed for a user is pre-processed as described herein (e.g. by low pass filtering and by selecting data captured at times when the data is expected not to be of poor quality on the metrics described above). The resulting data forms a signature of the user's gait. That signature can be compared with one or more gait signatures previously collected for the same user. These signatures can be used to detect information suggestive of certain conditions, and to help manage and monitor such conditions. These may be conditions with which clinicians do not currently use gait information. Differences in digital signatures captured over time may indicate non-specific changes to gait due to factors such as fatigue, weight loss/gain, breathlessness, joint or muscle pains, change in posture, neurological impairment with specific associations to cancers, cardiovascular/respiratory/metabolic conditions, musculoskeletal disorders or conditions affecting mental health.

Sensed gait information, or differences in sensed gait information over time may be used in any one or more of the following ways.

1. Information indicative of the user's current gait, or of a change in the user's gait from a baseline over time, may be analysed to estimate clinical factors of the type described above. Factors such as pain, or change in pain, may be estimated by analysing a gait signature alone or in combination with other data such as heart rate variability data for the user in question. Conveniently, change in a user's gait may be with reference to a baseline for that user. The baseline may be representative of the user's gait as sensed at a previous time, or as averaged over a period.

2. The user may have undergone surgery on a part of the body that has an influence on their ambulation (e.g. a hip, knee or ankle). In this situation it can be advantageous to monitor the user's recovery from the surgical procedure. The level of recovery may be correlated to aspects of gait such as length of stride, frequency of stride, the balance of either of the preceding factors as between steps taken with the left and right legs, or other factors indicative of the pattern of the user's gait. These may be sensed by one or more accelerometers. The system may monitor one or more of those factors as a user walks or runs, and compare the measured data against one or both of (i) predetermined reference data (.e.g. a consistent signature across a user population) and (ii) data previously measured for the same user. In dependence on that comparison the system may form an indication of the user's level of recovery. The data measured at a time may be compared to baseline data collected in respect of the gait of the same user at a previous time, or as averaged over a period.

3. Some individuals, for example some elderly individuals, are at increased risk of injury from falls. It would be desirable to be able to identify users at increased risk of falling so that steps may be taken to protect them, e.g. by education, physiotherapy or by adapting their home environment. The present system may form an indication of a user's risk of suffering a fall. That indication may be derived from information gathered over time about the user's gait as sensed by the system's one or more sensors. Factors that may provide input into the assessment include the regularity of the user's gait (the extent of variation in the period between strides), the extent of lateral motion of the user's upper body with each stride and the extent of twisting about a vertical axis of the user's upper body with each stride. The system may monitor one or more of those factors as a user walks or runs, and compare the measured data against one or both of (i) predetermined reference data (e.g. a consistent signature across a user population) and (ii) data previously measured for the same user. In dependence on that comparison the system may form an indication of the user's propensity to fall.

4. The system may be used to detect the presence of potential trip hazards in a user's environment. A user may have an increased chance of falling if they are exposed to uneven flooring, exposed cables, slippery floor mats or the like. The present system may monitor a user's gait so as to detect deviations from the user's normal gait pattern. Those deviations may, for example be in length of stride, frequency of stride or longitudinal acceleration of the user's upper body. Such deviations may indicate that the user has stumbled, or adjusted their stride to accommodate an unevenness in the ground. The system may store information about the locations at which those deviations take place. If the user is outdoors, the locations may be derived from a satellite location sensor. If the user is indoors the locations may be derived from any suitable indoor location system such as a radio beacon arrangement. The system assesses whether there is a tendency for the locations where deviations occur to be clustered in a similar location. If so, the system can form an indication of that location. Someone can then investigate whether there is a hazard at that location and if necessary rectify it. If such deviations are not clustered at a specific location then that may serve as an indication of a generalised propensity of the user to fall, which may be treated as described in the preceding paragraph.

5. As described above, data about a user may be gathered independently of what activities they are performing. Alternatively, a user may perform a standardised activity test. That may enable the system to make more precise predictions as to the user's wellbeing. Any suitable test may be used: for example a standard six-minute walk test. During the test data may be sensed by the sensors of the devices carried or worn by the user. The sensors may be configured to be operated at a higher-than-normal data acquisition rate during the test. Data indicating one or more of the user's heart rate, heartbeat regularity, respiration rate, respiration rate regularity, walking pace and gait metrics as described above during the test may be processed in accordance with a predetermined algorithm to form an indication of the user's propensity to heart failure, or of any build-up of fluid due to heart disease. Such an indication may be compared with a predetermined threshold (e.g. a consistent signature across a user population) or with data previously gathered for the same user to form an indication of risk or an indication of improvement or deterioration in the user's performance.

6. When data is being gathered, it is valuable to have a high level of confidence that the data being gathered relates to a specific user. If the sensors are intended to be carried or worn by a first user but are instead being carried or worn by a second user then it is desirable for that to be detected so that the data that is gathered is not attributed to the first user. One way in which this may be done is by monitoring factors indicative of the ambulatory gait of the user who is carrying or wearing the sensors. That may be done irrespective of whether the primary data that is to be collected includes gait data. Information about the first user's gait (e.g. its typical stride frequency, stride length, balance between left and right or acceleration pattern over the course of a stride) is sensed and stored. That constitutes reference gait data for the first user. As further data is subsequently being gathered information about the gait of the user can sensed and compared with the reference gait data. If the subsequently gathered data differs from the reference data by more than a predetermined amount then the system may conclude that the sensors are no longer being carried or worn by the first user. The primary data that is currently being collected may then be disregarded as an indication of the functioning of the first user. The reference data for the first user may conveniently be gathered during a standardised test, as described in the preceding paragraph. Reference gait data may be used for purposes other than improving data integrity, as described above. For example, it may be used to increase security, for example for financial applications or for physical access control. When a user wishes to access a resource they may authenticate themselves by means of a device such as device 1. An application running on the processor of that device, or an operating system running on the processor of that device, may form an indication of a level of confidence that the device is being operated by a given user. That indication may be formed in dependence on a comparison between reference gait data for that user and contemporaneously gathered gait data. The application or operating system may be configured to estimate the strength of the correlation between the reference gait data and the contemporaneously gathered gait data and to form an indication of a level of confidence that the device is being operated by the given user in dependence on that correlation, with the level of confidence being higher the better the correlation is determined to be.

When data is being collected from a body-carried or a body-worn sensor such as those described above, analytical techniques can be used to improve the reliability of the data being gathered. Examples of such techniques include the following:

1. Selection of a suitable sampling window can improve the reliability of the gathered data. The sampling window is the period over which a set of data is gathered for collective analysis. For example, heart rate or gait data may be gathered over the period of a sampling window, and that data may then be taken as being representative of the underlying function or feature, thereby providing a data point as to the value of that function or feature. In many conventional systems a sampling window of 10 seconds or more may be used. This may be thought to permit time-averaging to reduce potential errors due to possibly inaccurate instance of sampling. In contrast, it may be preferable to select a sampling window of 3 seconds or less. That may provide for greater accuracy by allowing the system to generate many separate data points relatively quickly, thus improving the accuracy of the solution and the classification rate. The actual length of the sampling window may be defined through a search-grid optimisation process. A sampling window may be selected by a mechanism such as that with a view to yielding the most accurate and rapid classification results.

2. The signal-to-noise ratio of the data gathered by a sensor may be improved by applying a low-pass filter to the data gathered by that sensor. The low-pass filter may be applied by means of a Fourier transform. Other noise filtering methods may be used to improve the quality and fidelity of the signals produced by the sensors before feeding the dataset to the classification algorithm.

3. When a user is carrying or wearing a sensor the system may have the capacity to collect data continuously. However, it may be advantageous for the system to selectively gather data at specific times when it is detected that the user's behaviour is appropriate to provide accurate data. For example, a carried device may be in a user's pocket at some times and in the user's hand at other times. Some factors (e.g. gait factors) may be sensed more accurately when the device is being carried in a non-hand-held manner, whereas other factors (e.g. the user's heart rate) may be sensed more accurately when the device is being carried in a hand-held manner. Various inputs may be used to estimate how a device is being carried: for example if the device is being used to make a phone call, or a user is providing input to a user interface unit of the device that may be taken to indicate that the device is currently hand-held. Then information may be sensed or not sensed; or sensed data may be gathered or discarded in dependence on the estimated carrying state of the device. Other states that could be estimated and in dependence on which data may be sensed or not sensed include whether the user is walking in a substantially straight line or alternatively in a curved direction; and whether the user is walking on a substantially flat surface or is climbing or descending. In general, information indicating the position and/or motion of a device may be used as an input to decide whether to gather or use data at that time. In that way data points of less reliability or relevance may be discarded before a sensed dataset is fed to a classification algorithm.

4. In a similar fashion to the preceding paragraph, a determination as to whether to gather or use data may be made in dependence on the orientation of a device. That may indicated whether the device is likely to be in a user's hand or alternatively in a pocket or handbag. The orientation of the device at the time data is sensed may be fed to a classification algorithm to improve the accuracy and speed of the results generated by the system.

Gait signatures detected by the present system may be used to derive an indication that a user might be suffering from a certain condition. Since gait abnormalities can result from multiple causes, it is expected that in practice clinical investigation will be needed to establish whether a condition indicated by the present system as being possible is actually present. Gait signatures detected by the present system may be used to monitor the state and/or progress (deterioration/improvement) of a previously diagnosed or suspected condition over time and/or the user's response to treatment of a disease. Some non-limiting examples of conditions that may be indicated, or whose progression may be monitored by the present system include: osteoporosis (changes in bone mineral density), depression, COPD, diabetes, early heart failure decompensation, cancer progression and relapse, rare conditions such as lysosomal storage disorders, falls risk and pain.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A data processing system comprising:
a processing device configured to collect sensed data representing the acceleration characteristics of a user's gait and associated locations and to process that data in accordance with a predetermined algorithm to (i) estimate instances in the acceleration characteristics that are indicative of uneven motion and (ii) identify locations within a predetermined distance of which more than a predetermined number of such instances have occurred and to output an indication of such locations.

2. A data processing system as claimed in claim 1, wherein the system is configured to identify said instances as being instances for which the acceleration data has either (i) a relatively high correlation with reference data representing uneven motion or (ii) a relatively low correlation with reference data representing even motion.

3. A data processing system comprising:
a processing device configured to collect sets of sensed data representing motion characteristics of a user's gait at a first time and at a second time subsequent to the first time, to process each set of sensed data to form a respective information set dependent on the user's gait at the first and second times, and to compare the first information set with one of (i) the second information set and (ii) a predetermined reference data set in accordance with a predetermined algorithm to identify a condition or change in condition of the user.

4. A data processing system as claimed in claim 3, wherein the condition comprises at least one of a level of pain and a level of recovery from surgery, a level of fall risk, and a likelihood of the user suffering from a predetermined disease.

5. A data processing system as claimed in claim 3, or wherein the sensed data comprises at least one of translational acceleration, rotational acceleration, translational speed, and rate of rotation.

6. A data processing system comprising:
a processing device configured to:
at a first time, collect sensed data representing motion characteristics of a first user gait and thereby form reference data representing a reference state of the user's gait; and
at a second time subsequent to the first time, collect sensed data representing motion characteristics of a second user gait, compare that data with the reference data and in dependence on that comparison form an indication of a level of confidence that the first user gait and the second user gait are of the same individual.

7. A data processing system as claimed in claim 6, wherein the system is configured to receive further sensed data, and to discard that data if said level of confidence is below a predetermined threshold.

8. A data processing system as claimed in claim 6, wherein the system is configured to govern access to a resource and to determine whether to provide access to that resource in dependence on whether said level of confidence is above a predetermined threshold.

9. A data processing system as claimed in claim 1, wherein the system is a mobile phone having a major display face and the system is configured to preferentially sense the sensed data when a normal to the major face is substantially horizontal.

10. A data processing system as claimed in claim 9, wherein the system is configured to preferentially sense the sensed data when a normal to the major face is within a predetermined angle of horizontal.

11. A data processing system as claimed in claim 1, comprising one or more sensors for sensing said sensed data.

12. A data processing system as claimed in claim 11, wherein the sensor(s) are packaged so as to be hand portable or wearable.

13. A data processing system as claimed in claim 12, wherein at least one sensor is provided with one of: a lanyard, a wrist strap, within an item of clothing, a body implant and an adhesive layer whereby the sensor can be attached to a user's skin.

14. A data processing system as claimed in claim 1, wherein the system is configured to aggregate sensed data over a window of 3 seconds or less to form an aggregated measurement and to apply the aggregated measurement as an input to said algorithm.

15. A data processing system as claimed in claim 1, wherein the system is configured to apply a low-pass filter to sensed data to form a filtered measurement and to apply the filtered measurement as an input to said algorithm.

16. A data processing system as claimed claim 1, wherein the system is configured to process at least some of the sensed data to estimate its correlation to predetermined data indicative of an activity of a user and to, in implementing said algorithm, apply a greater weight to data sensed at a time when that correlation is high.

17. A data processing system as claimed in claim 4, wherein the sensed data comprises at least one of translational acceleration, rotational acceleration, translational speed, and rate of rotation.
